(19) United States
(12) Patent Application Publication
(10) Pub. No.: US 2009/0203117 A1
(43) Pub. Date: Aug. 13, 2009

(54) Apparatus for Processing Biological Material

(76) Inventor: Lawrence Carl Crees, Upton Cheshire (GB)

Correspondence Address: John S Hale;Gipple & Hale, 6665A Old Dominion Drive, Mclean, VA, 22101, US

(21) Appl. No.: 12/084,120

(22) PCT Filed: Oct. 26, 2006
(86) PCT No.: PCT/EP2006/067838

§ 371 Date: Apr. 10, 2009

(30) Foreign Application Priority Data
Oct. 26, 2005 (GB) 0521851.6

(51) Int. Cl. C12M 1/34; C12M 1/26

(52) U.S. Cl. 435/287.3; 435/309.1

(57) ABSTRACT

The present invention relates to an apparatus for processing biological materials, comprising a platform having a plurality of sample receiving areas located thereon, each sample receiving area being substantially enclosed in a holding means, the apparatus also having at least one liquid carrier member mounted for supplying liquid to and/or removing liquid from the sample receiving area, and the platform and liquid carrier member being movable relative to one another so as to move sequential sample receiving areas into orientation with the liquid carrier member. The present invention is particularly suited to processing biological material (and in particular cell suspensions) for cytogenetic analysis.

The present invention relates to an apparatus for the processing of biological material and in particular, cell cultures grown in suspensions for subsequent cytogenetic analysis.

Advances in biochemistry and in particularly molecular biology and cytogenetics have resulted in reliable tests for certain types of diseases or conditions. Tests often require long and complex protocols, many of which are strictly time dependant. Most tests are performed on patient samples, such as biopsies, blood samples and other tissue samples for example. These samples need to be processed prior to analysis or further processing. Therefore, much of the time spent by clinical laboratory scientists is in processing samples, rather than analyzing the processed samples and producing a report of the results so as to enable the physician accurately to diagnose a disease or medical condition. Indeed, as such processing can be quite repetitive, tedious and requires a high degree of skill, there is the risk that a sample may be processed incorrectly, thereby rendering the laboratory analysis inaccurate, which may have serious consequences for the patient and the patient's family. Furthermore, a number of protocols involve hazardous chemical agents that necessitate the protocol being conducted in fume hoods/cabinets or require specialist protective clothing to be worn by the clinical laboratory scientist performing the procedure.

Common tests performed by clinical scientists engaged in cytogenetic analysis of clinical samples include karyotyping of individuals for predisposition to diseases or testing whether an individual condition is related to a chromosomal abnormality such as Down's syndrome. Immunostaining can show a range of conditions in cell samples such as excess endothelial cell growth in tumour samples. Fluorescent in situ hybridisation (FISH) and in situ hybridisation (ISH) can be used for many cytogenetic analyses including assessment of over expression of genes or proteins in cells. There are many more tests (and variations of those discussed) that are available for testing samples and these will be apparent to one skilled in the art. Most of these tests require some type of standardized cell processing and fixing, prior to the tests being performed on the sample which has been mounted or dried onto the surface of a slide. Such processing and fixing by its very nature often requires hazardous chemicals such as cross-linking agents or organic solvents that pose a health risk to the clinical scientist.

Commonly, during cytogenetic assays, of cells cultured by cell suspension techniques, the sample cells are first collected and cultured (for cell growth and division) in order to increase the amount of material available for testing. After culturing in a suitable culture medium, the cells are then harvested by first blocking mitotic spindle formation by the introduction of a metaphase arrest reagent (such as colcemid), that subsequently results in cell division stopping at the metaphase level (whereby chromosomes can be seen under a microscope). The cells can then be subjected to hypotonic treatment which increases their volume and renders the nuclear contents more predisposed to a monolayer disposition when dried onto a microscope slide. Prior to being placed on a slide for analysis or further processing steps, the cells (in suspension) are subjected to a fixative which acts both to remove water (in addition to some membrane material) from the suspension and also hardens the biological material prior to the suspension being placed on a slide. The reagents used in the processing of the cell suspension are often hazardous and the periodic separation of reagent from the cells in the suspension culture throughout the chromosome harvesting process involves the employment of centrifugation, vortexing and/or agitation steps. Accurate incubation times with the reagents used in the process are also required for the samples to be processed appropriately.

In order to address some of these problems outlined above and in order to allow the clinical cytogenetics laboratory to use the clinical laboratory scientist's time more effectively, a number of automated machines for processing of samples have been produced.

A number of devices have previously been disclosed, whereby biological samples are openly processed with reagents directly in a centrifuge. Whilst this apparatus reduces the manual input required to process biological material needing centrifugation steps, the unit is large and sample tubes have to be loaded manually into cluster buckets in the centrifuge one by one, in addition to possible contamination of the equipment should a tube break during processing (resulting in an unacceptable period of down time for the device). Such devices are currently available from ADSTEch (under the Hanabi™ brand, Japan) and Mires™, Italy.

It is an object of the present invention to address one or more of the problems associated with the prior art apparatus and methods of processing biological samples. It is also an object of the present invention to provide an apparatus for processing biological material that can simultaneously process multiple sample in an inventive manner. It would also be particularly advantageous should it be required that the process to be performed on significant volumes of samples having time dependant incubation protocols and requiring centrifugation. Furthermore, it is also preferred that an embodiment of the present invention provides an apparatus that can be used in relatively confined spaces and need not be placed in a fume hood or hazard biological containment cabinet.

According to the present invention, there is provided an apparatus for processing biological materials, comprising a platform having a plurality of sample receiving areas located thereon, each sample receiving area being substantially enclosed in a holding means, the apparatus also having at least one liquid carrier member mounted for supplying liquid to and/or removing liquid from the sample receiving area, and the platform and liquid carrier member being movable relative to one another so as to move sequential sample receiving areas into orientation with the liquid carrier member.

The present invention therefore provides for an apparatus that allows multiple biological samples to be easily processed in a single device. The present apparatus can be used in conjunction with a centrifuge and may also be automated or semi-automated. Furthermore, the present invention also reduces manual operator variability and allows trained and skilled operators to be utilized more effectively in the laboratory.

The term “platform” should be taken to encompasses a number of different structures such as discs or armed members and indeed any structures that would allow the apparatus to operate effectively. The sample receiving areas may in turn be removable so that a sample may be loaded outside of the apparatus. The term “liquid” should be taken to encompass liquids containing solid material, such as liquids containing cross-linked material or cell membrane debris for example.

The apparatus may further comprise a centrifuge for centrifuging one or more of the sample receiving areas. The sample receiving area may comprise a tube. The holding means may comprise a tube holder that may in turn hold a plurality of tubes. It will be apparent to one skilled in the art that the tube holder may accommodate bespoke or standard laboratory tubes that can be placed in a centrifuge. If required, the holding means may be adapted or adjusted so as to receive tubes of differing sizes and dimensions. The holding means will preferably comprise a vessel having an opening disposed therein, wherein a closure means is located relative to the opening for the selective opening and closing of the vessel. In order to allow access to the interior of the vessel, it is preferable that the closure means can be actuated by the apparatus automatically (when access is required) and/or manually (when access is required by the operator). It therefore follows that tubes (or other sample containers) can be placed in the holding means when the vessel is open and the vessel effectively sealed (when closed) from the environment when required, such as when a sample is being incubated with a hazardous reagent, or the holding means is being centrifuged.

Preferably, the closure means comprises two complementary covers that are biased (such as by means of a spring) to a closed position. The opening of the vessel may be due to tabs/projections located on the complementary covers being moved apart. Therefore, an operator or the apparatus itself can open the vessel by pushing/pulling the tabs apart and thus gain entry to the sample receiving areas (which may comprise a number of removable tubes). Furthermore, the holding means may be adapted to be received on the platform and/or the centrifuge. The holding means may be adapted by the provision of topographical features that interact/releasably engage features provided on the platform and/or the centrifuge. For example, the platform may have one or more receiving structures disposed thereon for receiving one or more holding means. Preferably, the receiving structures are disposed radially on the surface of the platform. If required, the platform can be used as the centrifuge platform itself or a separate centrifuge platform may be provided. It will also be evident that should the platform not be used as the centrifuge platform, the sample receiving areas may be processed by means of the liquid carrier members simultaneous to the centrifugation of other sample receiving areas. The holding means may also feature internal dividing walls constructed so that when the closing means is in the (default) closed position, sample integrity is maintained, aerosols generated during sample processing steps are contained and the possibility of cross-contamination between sample receiving areas is removed in the event of a spilled or shaken holding means.

The apparatus further may comprise a movement means for moving one or more holding means from the platform to the centrifuge. Such a movement means may comprise a picking arm, which are commonly known in the art. Should such an arm be employed, then it will preferably use a topographical feature located on the exterior of the holding means in order to move the holding means between the platform and the centrifuge. The apparatus may further comprise a bowl for containing the centrifuge and it is preferred that the bowl is made from a suitably hard material such as a metal or alloy so as to prevent objects flying out from the centrifuge in the event of a breakage, failure or malfunction.

The apparatus further may also comprise a support that is adapted to receive the platform. Such a support may be capable of sliding from a “loading” position (whereby an operator can load the platform into the apparatus) and a “processing” position (whereby the apparatus may process the samples). The support may be manually or automatically actuated and may also form a cover that can additionally be used as a cover for the centrifuge, when in operation.

The liquid carrier member may comprise a plurality of actuatable tubes and tube carriers capable of dispensing reagents and/or removing waste from one or more samples located in one or more sample receiving areas. There may be provided a first tube or first plurality of tubes for dispensing reagents and a second tube or second plurality of lubes for removing waste material and these tubes will be connected to reagent bottles/reagent mixing stations and waste bottles etc. Additionally, the liquid carrier may further comprise an actuation means for opening the complementary covers so as to gain access to the sample receiving areas within the holding means. The actuation means may comprise one or more means capable of moving the labs or lugs on the complementary covers apart when the means are brought into contact with the tabs of the complementary covers so as to allow entry to the holding means. The means of the actuation means may comprise spears or pivotable levers. In order to achieve smooth and consistent opening of the complementary covers and accurate transfer of liquid to the sample receiving areas, the liquid carrier may move in a substantially vertical plane relative to the sample receiving area. As previously described, the vessel of the holding means may have one or more topographical features disposed thereon for positive engagement with the platform and/or the centrifuge and/or the actuation means of the liquid carrier.

As some protocols will require a solution to be agitated (for example for the re-suspension of cells in a reagent), the apparatus may further comprise a means for agitating the sample receiving areas. It is also preferred that the platform is rotated by means of a Geneva wheel.

The apparatus may be contained within a substantially sealed housing when in operation. The interior may be accessed by a transparent/semi-transparent door, which may for example be made from a polycarbonate material. The reagents and wastes may also be accessed by a door in the apparatus, in order that they can be serviced as and when required. Therefore, a number of parameters can be controlled within the housing when processing is taking place. For example, the atmosphere may be controlled in order to allow for correct drying rate of a centrifuged cell pellet or a resuspended cell pellet if necessary either between steps in a protocol, or at the end of processing the material. The atmosphere may also be adapted to counteract the ambient temperature outside of the apparatus and indeed, the atmosphere of the apparatus may be controlled in such a manner that local environments near to, or around a sample or platform of samples may be controlled. The atmosphere of the whole apparatus (or near to the platform) may be controlled by adjusting one or more of the following variables: temperature, relative humidity, air pressure and volumetric air flow rate. These variables may be precisely controlled at the sensitive areas where the samples are held. The control of local environments may utilize an air conditioning means disposed near to or in conjunction with the liquid carrier member so that individual samples or reagent feeds can be subjected to the required local environmental conditions of a given protocol. It will be apparent to one skilled in the art that all of the variables can be controlled by currently available air conditioning apparatus (albeit a small scale apparatus) and the conditioned air can be supplied to the samples receiving areas, platforms or housing interior by pipes with baffles or similar diffusion devices if required. The air conditioning (including the drying of samples (if required)) may be automated and/or connected to a central processing unit. In order to assist in the automation of the air conditioning, sensors may be used to relay conditional data to the central processing unit to adjust the variables where appropriate. The central processing unit may adjust the variables depending on the protocol used and/or may be adjusted for bespoke operations or protocols.

Additionally, or alternatively, the sensor may comprise a turbidity monitor which is incorporated into the apparatus so as to assess a sample or batch of samples to ensure that all are subjected to the correct processing and processing conditions to support output of optimised quality from the subsequent slide dropping/drying process. The apparatus may also comprise a pellet monitor to assess the quantity of material in a pellet for optimum re-suspension in a given quantity of reagent. Preferably, the apparatus is located within a housing that may be substantially sealable. Furthermore, the housing may also allow any fumes from reagents to be contained substantially within the apparatus. The housing may also be connected to an air extraction means (which may include being placed within a fume cupboard) in order to remove potentially hazardous organic vapour fumes from the apparatus to an external or integral vapour outlet, which may itself comprise active materials employed as neutralizing (or “scrubbing”) agents. The air extraction means may also comprise a biological or microbiological contaminant fume treatment device (such as a HEPA filter) located integrally or external to the apparatus, commonly such devices are known as extraction and scrubbing and HEPA filter devices. It is preferred that the air flow is either passed or directed from the top of the device to the bottom of the device. The air flow may also be re-circulated within the housing. The fume treatment device may be an extraction fan which passes fumes over a fume absorbent material before allowing the treated air to be expelled into the surrounding environment and/or for further treatment. The fume absorbent material may be a catalyst or a material (such as a column of soda lime for example) which absorbs unwanted compounds such as organic vapors. The treated air may also be recycled back into the apparatus and used for air conditioning to assist drying for example and thus reducing the overall power input requirement of the apparatus. The reagents may also be held within the housing to allow for the apparatus to be a complete processing unit. Access to the reagents may be made via a separate door disposed on the housing in order to allow for replacement and washing of the apparatus.

It will be evident to one skilled in the art that a number of reagents may be used in the apparatus and the specific reagents will be dependant upon the protocol being implemented on the biological material. For example, in the case of processing of cell suspensions for cytogenetic analysis, may utilize three reagents: a cell cycle arrest reagent (such as colcemid), a hypotonic reagent; and fixative, in addition to washing steps if required. A given reagent may be previously mixed or may be prepared within the apparatus prior to being applied to the biological material, the latter being the preferred case with some reagents that do not have a long shelf life. The temperature of any given reagent may also be controlled by the apparatus. For example, should the samples be subjected to a fixation protocol, the fixative may be mixed by the apparatus for dispensing to the sample. Preferably, the required fixative is either automatically pre-mixed or mixed in-line immediately prior to dispensing the fixative in the appropriate ratios and quantities, depending on the sample size and type and harvesting process protocols and thereby processing the samples accurately. The constituents of the fixative may be defined by the user of the apparatus or automatically selected by the apparatus depending on the protocol being utilized. Furthermore, the reagents may be chilled or heated prior to dispensing according to harvesting process protocol requirements. There may be multiple washing steps in order to gradually elute a reagent from a sample. For example, multiple washing steps or pre-fixative steps may be required to remove cell membrane components, prior to applying a complete fixative reagent step to the sample. Similarly, multiple washing steps may be required to elute neat reagents from the sample. The reagents may be dispensed and/or the waste material removed by means of suitable pumps. Such pumps will preferably be electronically controlled so as to ensure the correct quantities of reagent are dispensed against a measured time interval. Preferably, the liquid carrier members are capable of dispensing a metered quantity of reagent to a sample and may provide for a single controlled drop of a reagent of a known quantity onto the sample. As previously described, the removing member may be connected to a waste tank located within the apparatus or connected to waste disposal device located outside the apparatus. Should the waste tank become full to capacity or become near to full capacity, an alarm be provided (e.g. to provide a sound or visual alert) so as to alert the user to the fact. Similarly, should the reagent feed vessels become either full to capacity or become emptied so that there is a substantial risk of the apparatus containing insufficient reagent stock to complete the process on the given number of samples and according to the user's selected process protocol then an alarm may be produced so as to alert the user to the fact. Alternatively, or additionally, an indicator relating to the quantity of waste material in the tank or the quantity of reagents held in feed bottles may be provided. It will also be obvious to one skilled in the art that should certain reagents be incompatible with one another (such that they react adversely together), separate waste tanks may be required.

The sample receiving areas and/or the holding means may have an identification means disposed thereon, such as a bar code, a dot code or a radio frequency emitter. The apparatus may further comprise a sensor for detecting the presence of a sample and/or identifying the sample. Preferably, the sensor is an optical sensor or RF sensor. Another example of a sensor for detecting the presence of a sample and/or identifying the sample is where the sensor may be a magnetic sensor. It will be apparent to one skilled in the art that a number of sensors may be utilized in the present apparatus, such as a reflective optical sensor or a scanning laser sensor to name two examples. The sensor may be provided in order to detect the characteristics of the processed biological material

The samples may have an identification means disposed thereon to assist the user in identifying the correct sample. Such an identification means may comprise a bar code, a dot code or a radio frequency microcircuit that uniquely identifies a sample. A suitable sensor for reading the identification means may be a laser scanner in the case of a bar code, or a radio frequency scanner in the case of radio frequency emitter for example. It will be apparent to one skilled in the art that the use of the identification means in addition to the a sensor for analyzing results will permit rapid and automated collation of data for a number of samples which can be printed off or linked to a sample database such as a Laboratory Information Management System (LIMS) for archiving, auditing and sample tracking purposes and/or further data analysis. Therefore the apparatus will greatly reduce the time and requirement of repetitive tasks undertaken by the clinical scientist to perform and monitor the processing and analysis of biological samples.

In order to increase the automation of the apparatus, it may additionally comprise an electronic control unit/central processing unit that controls the components of the apparatus. The components of the apparatus may include pumps, platforms, the liquid carrier member, linear and rotationally actuating arms, sensors and associated components. Such an electronic control unit may be programmable. The electronic control unit may be an EPROM (erasable programmable read-only memory) device. The sensors may relay information to the electronic control unit and may additionally interface with a printer and/or a computer. Therefore, the apparatus can be re-programmed with a bespoke operating protocol in accordance with different laboratories requirements. Such re-programming may be via a hand-held input device with a screen or alternatively may be via a computer linked to the apparatus. The computer may have a suitable user interface for ease of programming the apparatus and the computer may be connected to the apparatus by means of a standard cable types, such as USB, serial or parallel port cables.

It will be apparent to the skilled addressee that the apparatus may be used in the pre-analysis process for a number of different detection procedures for biological material such as FISH, ISH, Reporter molecules, Immunostaining, Cytogenetics, High-throughput screening of compounds for toxicology assay, cell culture, cell culture optimisation experiments etc. Furthermore, the apparatus may be used for processing the material in part of a protocol or indeed used for processing the whole sample when it is in a suitable format. Preferably, the apparatus is used for processing cells grown in suspension culture prior to preparing spreads of chromosomes on a slide from these cells. Processing material for cytogenetic analysis may involve processing suspension culture cells in order to analyze the associated chromosomes for abnormalities or for genomic or allelic variation etc. Commonly used techniques for cytogenetic analysis is via G/Q/R/T/C-banding and NOR silver staining, although other chromosome staining and banding techniques can also be utilized. It will be apparent to those skilled in the art that there will be new techniques yet to be developed may also be used in conjunction with the present apparatus. Usually, the cells analyzed will be in cells driven to the metaphase cell cycle prior to processing, although cells in other stages e.g. FISH assays on interphase cells may also be used.

The apparatus may refrigerate or heat some or all of the reagents so as to permit greater control over their reaction time with the sample. For example, fixatives may only be effective shortly after mixing and therefore if the apparatus is configured not to support in-line fixative mixing, the provision of refrigerating the fixative may allow the batch-mixed fixative to be used over a longer period of time.

It will be apparent to one skilled in the art that the apparatus as herein above described may be operably connected to a device for the preparation of a spread of chromosomes on a slide, so that the process of processing a biological sample (from a cell suspension) can be automated to high degree.

The present invention will now be described by way of example only with reference to and as illustrated in the following figures:

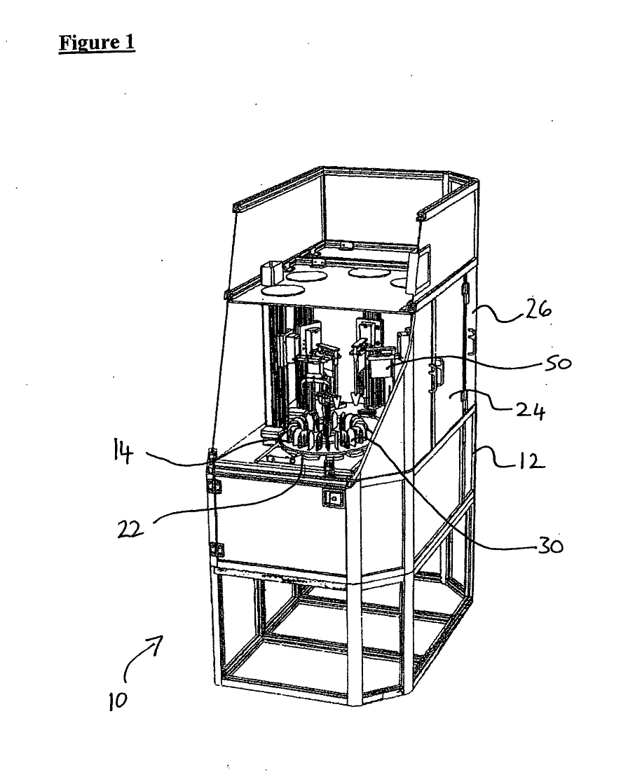

FIG. 1 is a perspective cut-a-way view of the apparatus in accordance with the present invention;

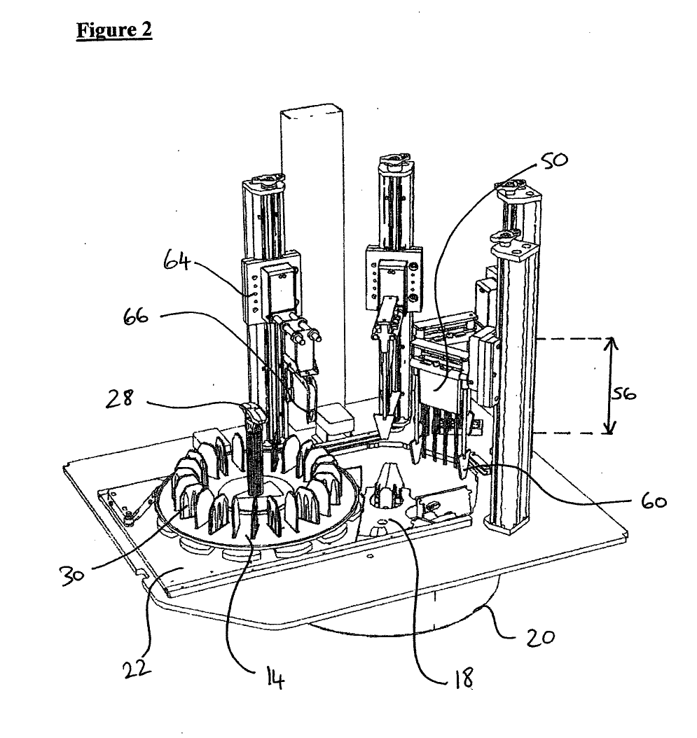

FIG. 2 is a perspective view of the sliding carousel within the apparatus;

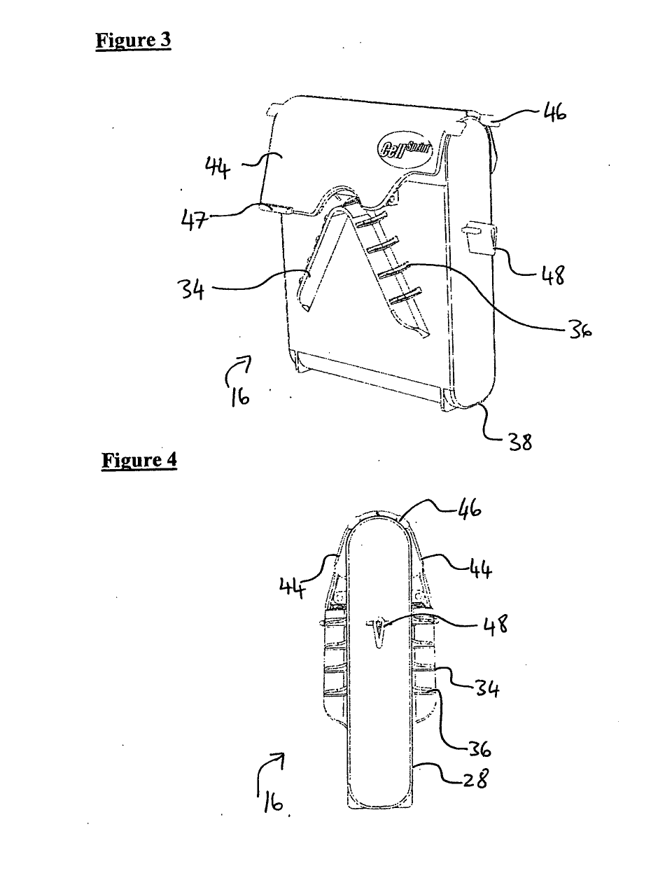

FIG. 3 is a perspective view of a tube holder used in the apparatus with the cover in a closed position;

FIG. 4 is a side view of the tube holder as shown in FIG. 3;

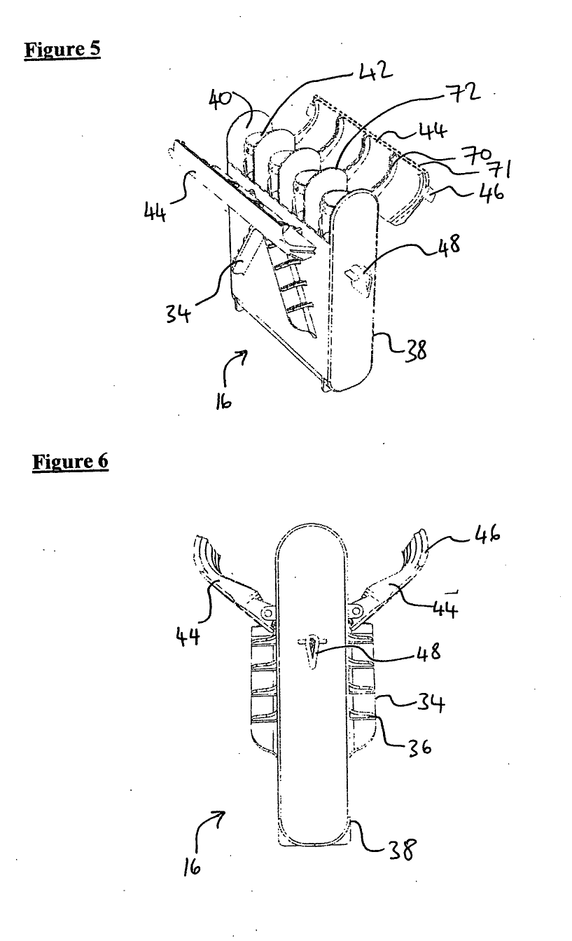

FIG. 5 is a perspective view of the tube holder with the cover in an open position;

FIG. 6 is a side view of the tube holder as shown in FIG. 5;

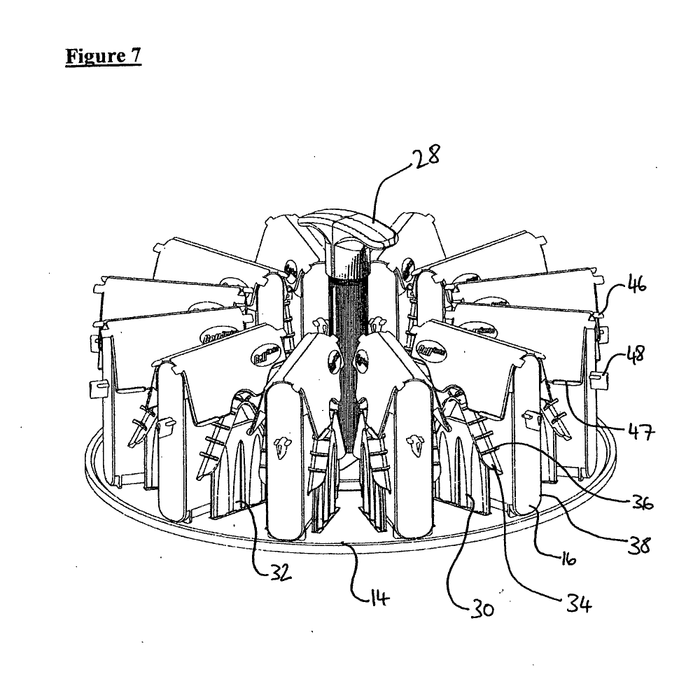

FIG. 7 is a perspective view of the carousel having a plurality of tube holders disposed thereon;

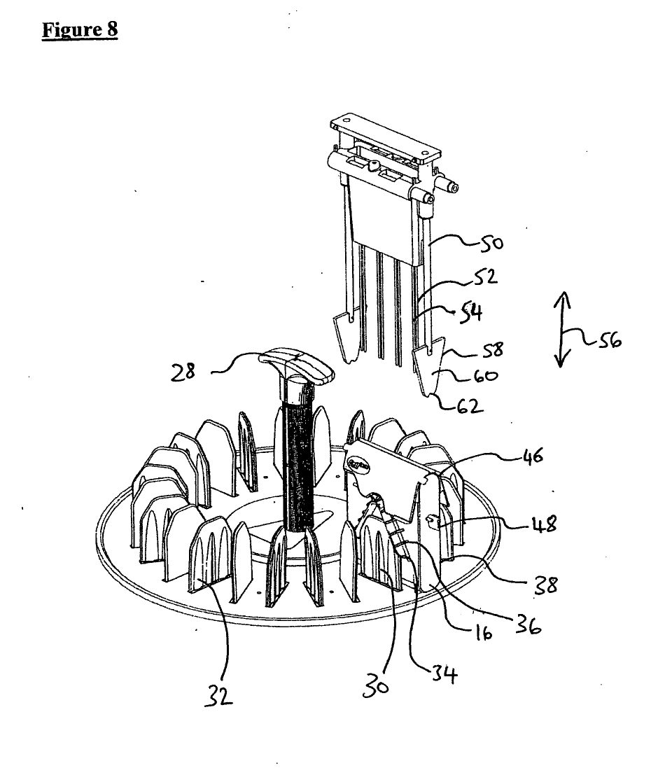

FIG. 8 is perspective view of a row of dispensing tubes positioned above a tube holder which in turn is located on the carousel;

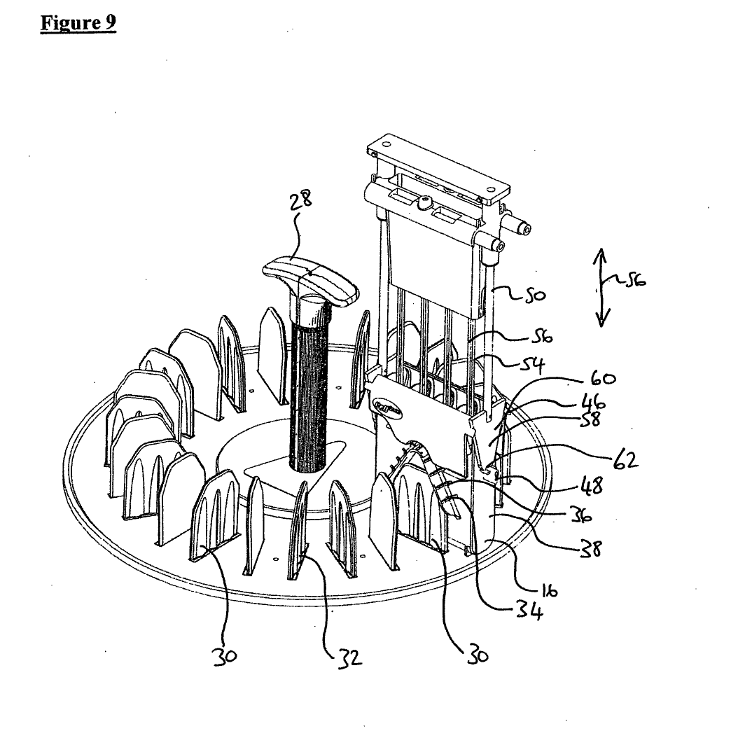

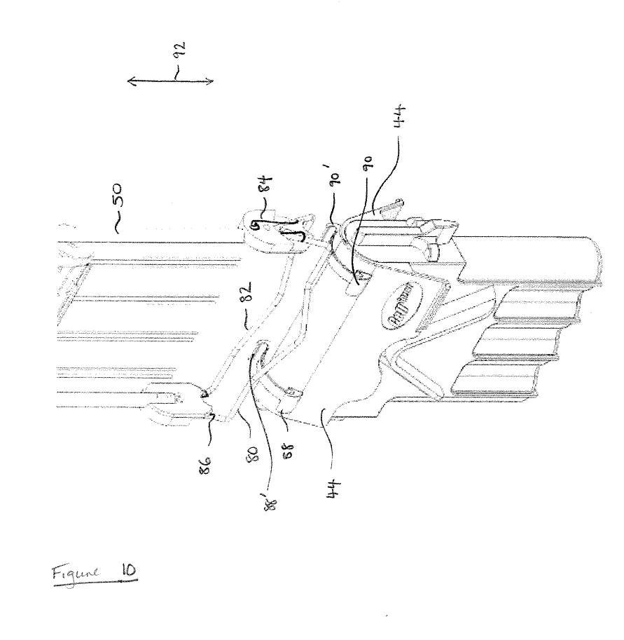

FIG. 9 shows a row of dispensing tubes being inserted into tubes located in a tube holder which is in turn placed on the carousel of the present invention;

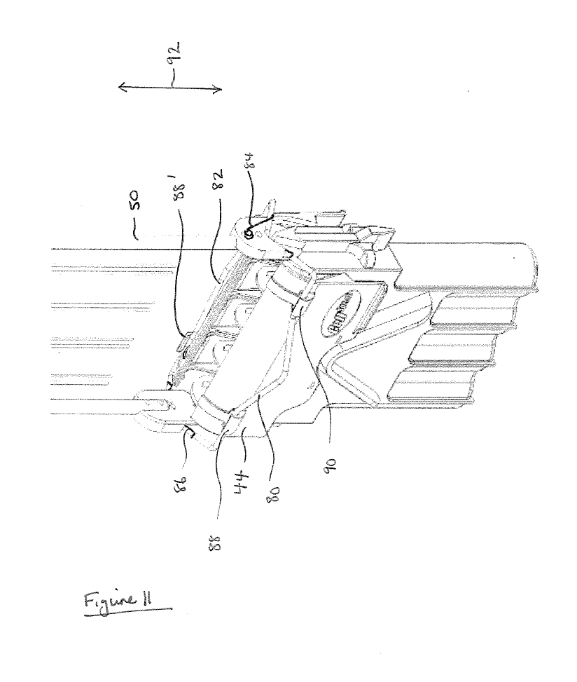

FIG. 10 is a perspective view of dispensing tubes positioned above a tube holder in a first configuration; and

FIG. 11 is a perspective view of the dispensing tubes and tube holder of FIG. 10 in a second configuration.

With reference to FIGS. 1 to 9, there is provided an apparatus 10 for processing biological material having a housing 12 within which is located a carousel 14 that is adapted to receive a number of tube holders 16. The housing 12 accommodates a centrifuge mechanism 18 which can spin the carousel 14 within the centrifuge drum 20. The carousel 14 is slidably mounted upon a slidable platform 22 which allows for positive location of the carousel 14 thereon. A pair of polycarbonate semi-transparent doors 24 are provided on the outside of the housing 12 in order to allow access to the interior of the apparatus 10 in addition to permitting the operator to see the apparatus in action. A reagent door 26 is also provided on the exterior of the housing 12 in order to allow reagents/radiant bottles (not shown) to be replenished and serviced as required.

The carousel 14 additionally has a central handle 28 for carrying the carousel about the lab and for easily locating the carousel on the slidable platform 22 as and when required. The carousel 14 additionally has a number of fins 30 which are radially configured such that two parallel fins are provided to form a locating member so that a tube holder 16 can be positively located thereon. The fins 30 are shown with three structural elements 32 which assist in providing strength to the fins. The fins are also shaped such that they can be received by two v-shaped locating members 34 disposed cither side of a tube holder 16. As with the fins 30, the v-shaped locating members 34 also contain a number of structural elements 36 in order to increase their strength.

The tube holder 16 has a housing 38 having four apertures which can accommodate a processing tube 42. The tube holder housing 38 has two complimentary “clam shell” covers 44 which are spring loaded such that they assume a closed position (as shown in FIGS. 3 and 4). On each end of a complimentary cover is a tab 46 which is used by the apparatus as a means of opening the covers so that access can be gained to the interior of the tube holder 16. The tabs 48 also protrude from the exterior of the tube holder housing 38 along with a locating member 48 that is used not only to positively locate the tube holder 16 when the tubes 42 are being processed, but also used to stabilize the tube holder 16. Furthermore, each tube holder 16 also has an aperture 40 for receiving a tube 42 and each aperture is self contained and compartmentalizes each tube when the complementary covers 44 are in a closed position, as lips 70,71 seal over the ridges 72 of the aperture 42. Therefore cross-contamination between tubes is reduced and should a tube break, then the liquid within the aperture can be removed and placed in a fresh tube without requiring the sample to be taken again.

The apparatus 10 also has a number of processing arms 50 which are connected to reagent and/or processing liquids (not shown) in addition to waste pipes etc. (also not shown). Each processing arm 50 has two tubes 52, 54, one of which is used to dispense reagents etc., whilst the other tube is used to remove excess or the fluid from a biological sample held within a processing tube 42. The arrangement of the processing arms are configured such that multiple dispensing and waste removal tubes 52, 54 can process multiple processing tubes 42 in a single operation. The movement of a processing arm 50 in a vertical plane 56 allows access to the interior of the tube holder 16 by means of the tabs 46 on the complimentary covers 44 riding over the inclined surface 58 of two spade shaped elements 60, thereby pushing the spring-loaded complimentary covers 44 apart. It follows that when the processing arm 50 is raised, the complimentary covers 44 automatically close as the spade shaped elements 60 are smoothly removed from contact of the tabs 46 located on the tube holders 16. Furthermore, at the base of the spade shaped element 60 is a notch 62 which positively locates the locating member 48 of the tube holder housing 38 when the spade element 60 is in place between the tabs 46 of the complimentary covers 44. Alternatively, each processing arm is used to dispense a given reagent or to remove waste material as and when required.

In use, the apparatus is used for processing cell suspensions of biological material in an interim processing step for the cytogenetic analysis of a given biological sample. However, the apparatus could also be used in other fields of biological research, where processing of biological samples by means of liquid reagents are used. The apparatus 10 may be free-standing or alternatively be located on a lab bench or indeed in a fume cupboard depending upon its configuration. Whilst not shown in the FIGS., the apparatus additionally has a vacuum pump and HEPA filter which is ducted from the bottom to the top of the apparatus in order to remove and address any potentially harmful fumes which may be omitted from the reagents and/or the processing steps. As previously described, the apparatus additionally comprises a number of reagents/waste vessels (not shown) which are located behind the reagents door 26 which supply the dispensing and removing tubes 52, 54 of the processing arms 50 with suitable reagents and vessels within which waste material can be deposited.

The reagents are held in reagent vessels (not shown) into which supply tubes are inserted which are connected to suitable pumps that further supply the dispensing tube tips with reagent when required. The peristaltic pumps are also controlled electronically by the electronic control device. A waste vessel is also provided in order to receive waste material from the removal tubes, which are also connected to suitable pumps and controlled by the electronic control unit.

As the carousel 14 can be removed from the apparatus, it will usually be loaded by a laboratory technician outside of the housing 12 by means of placing processing tubes 42 having a given sample being placed within an aperture 40 of a tube holder 16. In order to open the tube holder, the two linger grips 47 are pressed together, resulting in the opening of two complementary covers 44. Each position on the carousel 14 is numbered in addition to each location of a processing tube (which can only be inserted on the carousel in a certain orientation), so that the technician knows precisely which sample relates to which processing tube. Alternatively or additionally, each tube holder 16 may be provided with a bar-code which can be easily read by an external device, or indeed the apparatus 10 itself for the positive identification of an individual and/or batch of samples. The use of an RFID can also be implemented in order to ensure accuracy of the sample locations etc. When the carousel 14 is completely (or indeed partially) loaded with tube holder 16, it is then placed on the slidable platform 22. As previously discussed, the slidable platform 22 has a locating means by which the carousel can only be inserted in a certain manner so that the apparatus 10 knows the precise location of each tube holder 16.

When the carousel 14 is in place in the apparatus, the biological samples are processed according to a given protocol which will be computer controlled (not shown) and that is defined by the user. Therefore, parameters such as incubation time with reagents, centrifuge speeds and times, agitation and reagent sequences can be defined and implemented using the apparatus. Therefore, once the apparatus has been loaded with the carousel, the protocol can be invoked by the operator of the apparatus and all necessary processing steps undertaken subsequently and automatically by the apparatus.

During the processing of a biological sample, the carousel 14 can be rotated about an axis defined by the handle 28 so that each processing arm 50 can process biological samples held within processing tubes 42 of a given tube holder 16. In practice, the processing arm 50 can move in a vertical axis 56 depending on whether the processing arm 50 needs to gain entry to a tube holder 16.

When the processing arm 50 moves in a downward direction towards the tube holder 16, the tabs 46 ride over the inclined surface 58 of the spade shaped elements 60 and therefore the complimentary covers 44 are opened against the spring mechanism (not shown) holding the covers together. The notch 62 disposed at the end of the spade shaped elements 60 positively locates with the locating member 48 of the tube holder housing 38 which acts to stabilise the tube holder 16 during processing steps. However, due to the arrangement of the v-shaped locating member 34 and the fins 30, slight movement is permitted. A reagent may therefore be dispensed by means of a dispensing tube 54, or waste fluid removed by means of a waste tube 56 sucking the waste material from the processing tube 42. If required, the tube holder may be agitated by means of a movement by the spade shaped element 60 or by means of an additional device touching the tube holder 16 either directly or indirectly, should it be required. When the biological sample has been subjected to the relevant processing step, the processing arm is automatically raised and in doing so, the spade shaped elements are removed from contact of the tabs 46 of the complimentary covers 44 and thereby the springs allow to bring the covers together and the tube holder being substantially closed.

Should a centrifuge step be included in the processing steps of the biological material, then the carousel 14 may be lowered into the centrifuge drum 20 whereupon centrifuge mechanism 18 may be actuated in order to exert a force on the tube housing 16. Alternatively, a picking arm 46 may be used to remove the tube holder 16 from the carousel 14 so that it can be loaded directly into the centrifuge mechanism 18. The picking arm 64 has two claws 66, both of which have an aperture disposed therein for receiving the locating member 48 of an individual tube holder 16 and therefore each tube holder can be individually picked from the carousel 14 and loaded directly onto the centrifuge mechanism 18. Each tube holder 16 has a v-shaped member 34 that can receive cylindrical shaped protrusions located in the centrifuge. When the centrifuge starts to spin, the tube holder 16 can move about the protrusion so as to assure a horizontal position. It follows that when spinning is finished, the tube holder 16 resumes a vertical position. The slidable platform 22 additionally provides a “cover” for the centrifuge mechanism so as to shield a laboratory technician. However, as the centrifuge mechanism 18 is located within a steel drum 20, a cover is not necessarily required within the self contained apparatus. After a centrifuge step, the picking arm 64 is then used to place each tube holder 16 back on the carousel 14 in the correct position and further processing steps completed as necessary.

In an alternative embodiment processing arms 50 have a pair of pivoting levers 80, 82 biased into a closed configuration (FIG. 10) by springs 84, 86. Each lever 80, 82 is received by a pair of lugs 88, 90 and 88′, 90′ respectively, which are disposed on “clam shell” covers 44. Downward displacement of arms 50 in a vertical plane 92 allows access to the interior of the tube holder 16: levers 80, 82 engage with lugs 88, 90 and 88′ 90′, and once the bias of springs 84, 86 and the spring mechanism (not shown) holding the covers 44 together has been overcome, levers 80, 82 pivot into their open configuration (FIG. 11) pushing the spring-loaded covers 44 apart.

As shown in the diagrams, each carousel can contain twelve tube holders containing four samples each and therefore seventy two samples may be processed at any given time. Indeed, a number of tube holders may be located within the centrifuge mechanism and indeed being subject to the relevant centrifugal forces, whilst other tube holders are being subjected to reagent steps. Indeed, the tube holders 16 may also be processed directly within the centrifuge mechanism 18, should it be required.

Due to the configuration of the tube holder 16 and in particular the complimentary covers 44, processing lubes 42 are self-contained within the tube holder housing 38 and this further reduces the risks of spillage etc. to the laboratory technician. It will be obvious to one skilled in the art, that often during centrifugation, sample tubes often split resulting in the apparatus being unusable until thoroughly cleaned, and should a processing tube 42 break within the tube holder 16, the apparatus 10 is saved from such a spillage as the spillage will be held within the holder itself.

The processing tubes 42 may be bespoke sizes, or alternatively standard tube sizes currently used in laboratory environments. If required, the tube holder housing 38 could be adapted in order to receive different sizes and shapes of processing tubes 42, although care would no doubt have to be taken so that the centrifuge mechanism 18 was correctly weighted. Furthermore, the processing arms 50 and/or picking arm 64 may be connected to a collision detection system, whereby should the arms not be correctly located relative to the tube holders, an error is reported to the laboratory technician and the apparatus stopped in order to prevent any damage being occurred thereto. The internal conditions of the apparatus may also be controlled such as humidity and temperature in order that each biological sample is processed in the same manner and at optimum conditions. The carousel may be rotated within the apparatus by means of a “Geneva” wheel or alternatively by any prior art mechanism which will be apparent. The processing arms, and by preference, the arm performing reagent aspiration after each respective centrifugation step, may also be raised, rotated and lowered so as to access an online wash station in order to wash the reagent tubes and tube carrier tips in order to maintain the integrity of each cell culture and to remove and possibility of “carryover” or cross-contamination between cultures in tubes sequentially processed, the substance of the wash material being variable according to the reagents involved in any particular stage of the process.

The apparatus is controlled by a built in program held in a electronic control device, which is re-programmable if necessary and such an electronic control device may have a number of protocols from which to choose from. The user may enter their own protocol as wished prior to a batch being processed, via the keyboard (not shown) which is linked to the electronic control unit. Alternatively, the electronic control unit may be linked to a computer via an interface so as to re-program the apparatus. After the apparatus has been loaded with the number of samples to be processed, the process can be initiated by starting the selected protocol (by selecting it on a keyboard linked to the electronic control unit (not shown)). After the completed protocol has been applied to each sample the machine automatically stops, and if necessary alerts the laboratory technician.

As previously stated, the apparatus will commonly be used for processing biological material used in cytogenetic analysis and with this regard, the reagents used may include a colcemid solution, a hypotonic solution and a fixative, such as a mixture of methanol and acetic acid). Due to the relative short shelf life of such reagents (which often require mixing prior to application, the apparatus may also provide for the mixing of various reagents at the processing arm 50 if required.

In this application, the apparatus is used in the intermediate step of preparing suspension cell cultures driven to cell cycle metaphase arrest in the cell cycle. Cells commonly used for cytogenetic analysis include those derived from fibroblast, placental mesodermal and amniocyte, blood, bone marrow, lymph node and solid tumour tissues, cells. The protocol was used to prepare cell cultures prior to fixing them on slides and before applying a process to highlight structural features within chromosomes (such as subjecting a protocol to G-Band the chromosomes). The apparatus is substantially leak proof, and in addition the main logic and control circuits together with most of the actuators are mounted in a compartment separated from the liquids providing for a reliable apparatus.

1. An apparatus for processing biological materials, comprising a platform having a plurality of sample receiving areas located thereon, each sample receiving area being substantially enclosed in a holding means, the apparatus also having at least one liquid carrier member mounted for supplying liquid to and/or removing liquid from the sample receiving area, the platform and liquid carrier member being movable relative to one another so as to move sequential sample receiving areas into orientation with the liquid carrier member and a centrifuge for centrifuging at least one of the sample receiving areas.

2. (canceled)

3. An apparatus as claimed in claim 1, wherein the platform is capable of rotating relative to the liquid carrier member.

4. An apparatus as claimed in claim 1 wherein the sample receiving area comprises a tube and said holding means comprises a tube holder.

5. (canceled)

6. An apparatus as claimed in claim 1, wherein said holding means comprises a vessel defining an opening thereon, and a closure means is located relative to the opening for the selective opening and closure of the vessel.

7. (canceled)

8. An apparatus as claimed in claim 6, wherein the closure means comprises two complementary covers that are biased toward a closed position.

9. (canceled)

10. (canceled)

11. (canceled)

12. (canceled)

13. An apparatus as claimed in claim 1 wherein the platform is used as the centrifuge platform.

14. An apparatus as claimed in claim 1 wherein the apparatus further comprises a movement means for moving one or more holding means from the platform to the centrifuge.

15. An apparatus as claimed in claim 14, wherein said movement means comprises a picking arm.

16. (canceled)

17. (canceled)

18. (canceled)

19. An apparatus as claimed in claim 1, wherein the liquid carrier member comprises a plurality of tubes capable of dispensing reagents and/or removing waste from one or more samples located in one or more sample receiving areas.

20. An apparatus as claimed in claim 19, including a first tube for dispensing reagents and a second tube for removing waste.

21. An apparatus as claimed in claim 1 wherein the liquid carrier further comprises an actuation means for opening the complementary covers so as to gain access to the sample receiving areas within the holding means.

22. An apparatus as claimed in claim 21, wherein the actuation means comprises one or more spears capable of moving tabs formed on the complementary covers apart when the spears are brought into contact with the tabs of the complementary covers.

23. (canceled)

24. (canceled)

25. An apparatus as claimed in claim 1, wherein the apparatus further comprises a means for agitating the sample receiving areas.

26. An apparatus as claimed in claim 1, wherein the platform is rotated by means of a Geneva wheel.

27. (canceled)

28. (canceled)

29. An apparatus as claimed in claim 1, wherein the sample receiving areas and/or the holding means have an identification means disposed thereon.

30. An apparatus as claimed in claim 29, wherein the identification means comprises one or more of a group consisting of a bar code, a dot code or a radio frequency.

31. An apparatus as claimed in claim 1, wherein the apparatus further comprises a sensor for detecting the presence of a sample and/or identifying the sample.

32. (canceled)

33. (canceled)

34. An apparatus as claimed in claim 1, wherein the apparatus further comprises a turbidity monitor for assessing the turbidity of a sample.

35. An apparatus as claimed in claim 1, wherein the apparatus further comprises a pellet monitor to assess the quantity of material in a pellet for optimum re-suspension in a given quantity of reagent.

36. (canceled)

37. (canceled)

38. (canceled)

39. An apparatus as claimed in claim 1, wherein the apparatus is used for processing biological material used in cytogenetic analysis.

40. An apparatus as claimed in claim 1, wherein the apparatus is used for processing cell cultures prior to preparing a spread of chromosomes on a slide.

41. An apparatus as claimed in claim 1, wherein the apparatus is connected to a device for the preparation of a spread of chromosomes on a slide.

42. (canceled)

43. (canceled)

44. An apparatus for processing cell suspension for cytogenetic analysis, comprising a platform having a plurality of sample receiving areas located thereon, each sample receiving area being tubular and substantially enclosed in a tube holding means, the apparatus also having at least one liquid carrier member mounted for supplying liquid to and/or removing liquid from the sample receiving area, the platform and liquid carrier member being movable relative to one another so as to move sequential sample receiving areas into orientation with the liquid carrier member and a centrifuge for centrifuging at least one of the sample receiving areas and sensor means mounted to said platform to detect the presence of sample and identify said sample in at least on sample receiving area.
